(19) United States
(12) Patent Application Publication
(10) Pub. No.: US 2026/0207500 A1
(43) Pub. Date: Jul. 23, 2026

(54) METHOD FOR PROVIDING A LIPOSOME SOLUTION FOR FORMATION OF MICROVESICLES

(71) Applicant: DEMCON curix B.V., Enschede (NL)

(72) Inventors: Gerritdina Marieke OLSMAN, Enschede (NL); Peter Johan Anton FRINKING, Enschede (NL)

(73) Assignee: DEMCON curix B.V., Enschede (NL)

(21) Appl. No.: 19/137,973

(22) PCT Filed: Dec. 14, 2023
(86) PCT No.: PCT/EP2023/085912

§ 371 Date: Jun. 11, 2025

(30) Foreign Application Priority Data
Dec. 20, 2022 (NL) 2033770

(51) Int. Cl. A61K 9/1271; A61K 9/1277

(52) U.S. Cl. CPC A61K 9/1271; A61K 9/1277

(57) ABSTRACT

The present invention relates to a method for providing a liposome solution comprised of a phospholipid blend for formation of microvesicles. The present invention further relates to a liposome solution obtained by the method for forming microvesicles, and the microvesicles comprised of the liposome solution. Furthermore, the present invention relates to the use of the liposome solution or the microvesicles as drug delivery enhancing agents or as drug delivery vehicle of drugs to a patient.

The present invention relates to a method for providing a liposome solution comprised of a phospholipid blend for formation of microvesicles. The present invention further relates to a liposome solution obtained by the method for forming microvesicles, and the microvesicles comprised of the liposome solution. Furthermore, the present invention relates to the use of the liposome solution or the microvesicles as drug delivery enhancing agents or as drug delivery vehicle of drugs to a patient.

Microvesicles (also known in the art as nano/microdroplets, microcapsules, microbubbles) are heterogeneous membrane-bound objects having a core, such as a fluid (i.e., gas or liquid), that is enclosed by the outer membrane, preferably a lipid bilayer membrane, in the case of lipid-stabilized droplets, or more preferably a lipid monolayer membrane, in the case of lipid-stabilized gaseous microbubbles. Microvesicles are typically produced in a dedicated system from a liposome solution comprising phospholipids to generate microvesicles. Such microvesicles can be used as drug delivery enhancing agents, for example by intravenous co-administration with parental drugs in the circulatory (blood) system for the treatment of the patient, or for parental administration of drugs or treatments to a patient, for example via intravenous administration of drug-loaded microvesicles in the circulatory (blood) system of the patient. For an effective drug delivery, it is important that said microvesicles have a predefined size and/or size distribution, wherein the distribution is preferably as narrow as possible.

Liposome solutions comprised of phospholipid blends are typically produced by dissolving the lipids in a solvent, and then reducing the volume either by lyophilization or distillation. Such liposome solution preparation methods to produce microvesicles make use of highly toxic carcinogenic solvents in an extensive multistep method, for example described in Segers et al. (Langmuir 2017, 33(39)). Production occurs in general according to a thin-film hydration method using toxic organic solvents, wherein lipids are mixed and dissolved in a mixture of chloroform and methanol at ambient pressure and a temperature of 60° C. or more. Then the pressure is reduced to evaporate the solvent. The resulting lipid film is then dried overnight in a vacuum oven at a pressure of 100 mbar and the next day, the lipids are rehydrated in a mixture of TRIS buffered water, propylene glycol (PG), and glycerin/glycerol again at 60° C. Directly after rehydration, the lipid dispersion is homogenized by sonication using, for example, a tip sonicator, and the dispersion is cooled down to room temperature.

Current methodologies to produce microvesicles make use of toxic carcinogenic solvents. A disadvantage of this procedure is that toxic solvents might be present in the end product. Posttreatments for removing traces of organic solvents are required, as well as additional clinical test for proving that the product is not toxic, in view of microvesicles formed from the liposome solutions and their subsequent drug delivery application. Moreover, sonication may introduce unwanted iron particles, which are shed from the sonicator, in the liposome solution. Furthermore, high concentrations of glycerol, typically 5 vol-%, result in e.g., formation of foam during stabilization after microbubble formation by microfluidic flow focusing due to gas dissolution. Furthermore, glycerol increases bulk viscosity which may negatively affect the process of microfluidic flow focusing. Glycerol is being used for rehydration of the lipids in the mixture, however resulting in an increase in viscosity of the liposome solution and decrease in high quality microbubbles produced by microfluidic flow focusing. A gas mixture and mixing device is needed to reduce foam formation during microbubble stabilization after formation of the microbubbles, resulting in long stabilization times (>1 h). Current methods are suitable for small, laboratory scale production, but often problematic upon scale-up to production-size quantities, including maintaining liposome solution (and resulting microvesicle) uniformity during the solvent removal step due to differential solubilities of the lipids, and minimizing solvent volume in view of reduced toxicity or improved environmental footprint of the production process.

Finally, elevated temperatures (>60° C.) are needed to obtain the liposome solution wherein the phospholipids are dissolved and to perform microvesicle formation, followed by immediate cooling for reducing microvesicle coalescence during microfluid formation at high flow rates, which is less sustainable, and the method is difficult to scale up due to the increase in energy costs. At present, hydration of phospholipids at lower temperatures results in a cloudy suspension with particles having a relatively large and non-uniform particle size distribution ranging from 0.1 μm to 10 μm. This suspension in turn cannot be filtered at lower (such as ambient) temperature when the suspension solution temperature is below the phase transition temperatures of lipids. The lipids would accumulate in the filters causing a restriction in the flow rate and eventually block the filters.

Considering the above, there is a need in the art for a simplified, large scale, more environmentally friendly, and more energy efficient method for the preparation of a liposome solution for use in the formation of microvesicles. Furthermore, there is a need in the art for a more environmentally friendly liposome solution for use in the formation of microvesicles.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a method for providing a liposome solution for formation of microvesicles, wherein the liposome solution is comprised of a phospholipid blend of at least two phospholipids having different phase transition temperatures (Tm), comprising the steps of
a) dissolving a first phospholipid having the lowest Tm in a pre-heated organic non-aqueous solvent having a temperature of below the Tm of the first phospholipid, providing a first liposome solution,
b) adding a second phospholipid in said first liposome solution to obtain a liposome dispersion, or
adding a second phospholipid in a pre-heated organic aqueous solvent having a temperature of below the Tm of the second phospholipid to obtain a second liposome dispersion,
c) maintaining the liposome dispersion at the temperature of below the Tm of the first phospholipid for 30 min to 2 hours, preferably 45 to 90 min, more preferably 60 to 75 min, preferably under stirring, to obtain a further liposome solution wherein the phospholipids are dissolved, preferably under stirring, or
maintaining the second liposome solution at the temperature of below the Tm of the second phospholipid for 30 min to 2 hours, preferably 45 to 90 min, more preferably 60 to 75 min, to obtain a second liposome solution wherein the phospholipids are dissolved, preferably under stirring,
d) adding of a preheated organic aqueous solvent having a temperature of below the Tm of the first phospholipid to the further liposome solution, preferably under stirring, or
adding the second liposome solution to the first liposome solution, preferably under stirring,
to obtain a pre-final liposome solution having an organic non-aqueous solvent to organic aqueous solvent ratio of 5 to 25 non-aqueous: 75 to 95 vol. % aqueous, preferably 7.5 to 20 non-aqueous: 80 to 92.5 aqueous vol.-%, even more preferably 8 to 15 non-aqueous: 85 to 92 aqueous vol.-%, most preferably about 10 non-aqueous: 90 aqueous vol.-%,
e) maintaining the pre-final liposome solution at the temperature of below the Tm of the first phospholipid for 2 to 12 hours, preferably 4 to 10 hours, more preferably 6 to 8 hours, preferably under stirring, to obtain a final liposome solution.

The method of present invention provides a liposome solution comprised of a homogenous lipid blend. Experiments show that the method of present invention for preparing the liposome solutions provides a more homogeneous liposome distribution resulting in more uniformity between microbubble and less coalescence in comparison to the known methods for preparing said solutions. Coalescence of microbubbles results in polydisperse microbubble populations. To maintain a monodisperse microbubble population coalescence should be avoided. The method is free of toxic carcinogenic organic solvents, so no further purifications steps are needed, and operates at relative low temperatures and is a simple procedure, the method is easy to scale-up whereas no excessive heating or cooling is required to obtain the liposome solution.

Liposomes are primarily composed of phospholipids, which are a diverse class of compounds composed of a hydrophilic head group covalently attached to a pair of hydrophobic fatty acids. Phospholipid molecules spontaneously form bilayers when added to aqueous solutions, as the phospholipids are driven to orient their head groups towards water and shield their fatty acid tails from it via the hydrophobic effect. Phospholipids can undergo phase transitions under the correct environmental conditions. The phase transition temperature (Tm) of the phospholipids refers to the temperature required to induce a change in the lipid physical state from the ordered gel phase to the disordered liquid crystalline phase. The main driving force of most phase transitions is temperature. Temperatures above the Tm of a lipid will transition lipids to a liquid phase, while colder temperatures will cause a transition to a solid-like phase. However, Tm can vary between lipids due to differing structural properties and strongly depend in environmental conditions (such as solvent and mixture conditions). Remarkable in the method of present invention in view of the known methods to produce liposome solutions is that lipids are dissolved in the organic solvent at relative low temperatures, more specifically below the Tm of said lipids. In comparison to known methods, increased volumes of non-aqueous organic solvent (preferably 7.5 vol. % or higher) is pre-heated to a temperature of below the Tm of the respective phospholipid.

The range of non-aqueous solvent is preferably larger than or equal to 7.5 vol. % based on the total volume of organic solvent in the liposome solution, to achieve an optimal dissolved lipid solution at the claimed temperatures. The mixture of organic non-aqueous solvent and organic aqueous solvent of the liposome solution comprises 5 to 25 vol.-% of organic non-aqueous solvent based on the total volume of the solution. Addition of the organic solvent to the first or second phospholipid results in that the lipid preferably fully dissolves in the organic solvent. However, due to the temperature being below the Tm of the lipid, time is required (preferably under stirring/gentle mixing) to fully dissolve the lipid. After addition of the lipid to the organic solvent, at least 90 mol-% of the lipid is dissolved, preferably at least 92 mol-% is dissolved, more preferably at least 95 mol-% is dissolved, even more preferably at least 99 mol-%, most preferably 100-% is dissolved within the disclosed time, providing a homogenous lipid blend.

According to a preferred embodiment, the present invention relates to the method, wherein the method further comprises step f) flow extrusion or filtering of the final liposome solution at a temperature below the Tm of the first phospholipid, preferably at 15 to 40° C., more preferably at room temperature of 18 to 25° C., thereby providing a final liposome solution for formation of microvesicles. Flow extrusion or filtering of the liposome solution may be performed using a sterilizing filter, preferably through a 0.2 μm polycarbonate/cellulose acetate membrane. This extrusion or filter step improves the liposome solution to provide a more uniform and monodisperse microvesicles. The method of present invention provides for microvesicles prepared at room temperature and rapid stabilization (<30 min.) and the product can be used rapidly after formation. In contrast to known methods for formation of microvesicles in the art, the present invention can produce microvesicles with the liposome solution of present invention with increased volumes of non-aqueous organic solvent by microfluidic flow focusing. The extruded final liposome solution can be for example stored at 4° C. for an extended period.

According to another preferred embodiment, the present invention relates to the method, wherein the temperature of the pre-heated organic non-aqueous or aqueous solvents is at most 20%, more preferably at most 10%, more preferably at most 5%, most preferably at most 1% below the respective Tm values of said first or said second phospholipids. The advantage of the method of present invention is that the lipids are homogenously mixed and dissolved at low temperatures, i.e., below the Tm of the phospholipids used in the lipid blend. The closer the temperature of the pre-heated solvent is to the Tm value of the phospholipid, the more efficient the process of dissolving the lipid into the solvent is achieved. However, when decreasing the temperature too much, no homogenous lipid blend can be achieved anymore. The method of present invention, using increased volumes of non-aqueous organic solvent (preferably 7.5 vol. % or higher), provides a phospholipid blend optimized for large scale production at low temperature.

According to yet another preferred embodiment, the present invention relates to the method, wherein the pre-heated organic non-aqueous solvent having a temperature of below the Tm of the first phospholipid in step a and the preheated organic aqueous solvent having a temperature of below the Tm of the first phospholipid in step d is 20 to 55° C. preferably 25 to 45° C., more preferably 35 to 39° C., most preferably 38° C.

According to another preferred embodiment, the present invention relates to the method, wherein the pre-heated organic aqueous solvent having a temperature of below the Tm of the second phospholipid in step b is 40 to 60° C. preferably 45 to 55° C., more preferably 48 to 50° C., most preferably 50° C.

According to a preferred embodiment, the present invention relates to the method, wherein addition of said preheated organic aqueous solvent or said second liposome solution in step d is performed in two to five separate steps, preferably three to four separate steps, wherein the added volume is divided equally among these steps. Addition in step d is preferably done in a multi-step fashion to reduce the chances or even prevent partial precipitation of the lipids and to obtain the liposome solution comprised of a homogenous lipid blend.

According to another preferred embodiment, the present invention relates to the method, wherein the final liposome solution comprises from 60 to 95 mol-% of the first phospholipid, more preferably from 70 to 90 mol-%, even more preferably 75 to 87 mol-%, most preferably from 80 to 85 mol-%. With the first phospholipid in the liposome solution being outside these mol-% ranges it was difficult to obtain a monodisperse microvesicles by microfluidic flow focusing.

According to a preferred embodiment, the present invention relates to the method, wherein the final liposome solution comprises from 5 to 40 mol-% of the second phospholipid, more preferably from 10 to 30 mol-%, even more preferably 13 to 25 mol-%, most preferably from 15 to 20 mol-%. With the second phospholipid in the liposome solution being outside these mol-% ranges it was difficult to obtain a monodisperse microvesicles by microfluidic flow focusing.

According to yet another preferred embodiment, the present invention relates to the method, wherein the first phospholipid is selected from the group consisting of 1,2-dipalmitoyl-sn-glycero-3-phosphocholine (DPPC), 1,2-distearoyl-sn-glycero-3-phosphocholine (DSPC), 1,2-dierucoyl-sn-glycero-3-phosphocholine (DEPC), 1,2-dimyristoyl-sn-glycero-3-phosphocholine (DMPC), 1,2-dimyristoyl-sn-glycero-3-phosphoglycerol (DMPG), 1,2-dioleoyl-sn-glycero-3-phosphocholine (DOPC), 1,2-dioleoyl-sn-glycero-3-phosphoethanolamine (DOPE), 1,2-dioleoyl-sn-glycero-3-phospho-L-serine (DOPS), 1,2-dipalmitoyl-sn-glycero-3-phosphoglycerol (DPPG), 1,2-distearoyl-sn-glycero-3-phosphoglycerol (DSPG), egg phosphatidylcholine (EPC), and hydrogenated soybean phosphatidylcholine (HSPC), preferably DSPC, more preferably DPPC. DSPC and DPPC are mostly used for current ultrasound contrast agents and have good safety profile in patients. DPPC is more preferred because of its low Tm values and smaller liposome size.

According to a preferred embodiment, the present invention relates to the method, wherein the second phospholipid is a phospholipid poly ethylene glycol (PEG) conjugate, preferably selected from the group consisting of N-(Carbonyl-methoxypolyethylene glycol(750-10000))-1,2-distearoyl-sn-glycero-3-phosphoetanolamine (DSPE-mPEG(750-10000)), N-(Carbonyl-methoxypolyethylene glycol(1000-5000))-1,2-dipalmitoyl-sn-glycero-3-phosphoetanolamine (DPPE-mPEG(1000-5000)), preferably DPPE-mPEG2k, more preferably DSPE-mPEG5000, most preferably DPPE-mPEG5000. The PEG chains molecular weight may vary from about 750 to about 10000 Daltons in the phospholipid.

According to a preferred embodiment, the present invention relates to the method, wherein phospholipids, more specifically the sum of the first and second phospholipids, are present in the final liposome solution at concentration of 1 to 25 mg/ml, preferably 5 to 20 mg/ml, more preferably 10 to 15 mg/ml. Concentration of the phospholipids within the claimed ranges are beneficial for microbubble formation by microfluidic flow focusing. Due to such high concentration of lipid vesicles in the liposome solution coalescence of microbubbles is strongly reduced or even absent. A high concentration of lipid vesicles is needed to prevent coalescence. What is surprising is that with the liposome solution of the present invention prepared at low temperature it was possible to form monodisperse microbubbles by microfluidic flow focusing.

According to yet another preferred embodiment, the present invention relates to the method, wherein the non-aqueous organic solvent is one or more selected from the group consisting of propylene glycol, ethylene glycol, preferably propylene glycol.

According to another preferred embodiment, the present invention relates to the method, wherein the aqueous organic solvent is one or more selected from the group consisting of phosphate buffer saline (PBS), buffered aqueous solution, saline solution, preferably PBS.

According to a preferred embodiment, the present invention relates to the method, wherein the method or liposome solution is free from glycerol or Diphenylphosphoryl azide (DPPA) and toxic carcinogenic organic solvents, such as chloroform. Advantageously, the liposome solution comprises no DPPA. Phospholipid compositions prepared via the prior art processes are difficult to extrude or to sterilize by filtration at low temperatures Furthermore, in view of the use of the liposome solution or the microvesicles as drug delivery enhancing agents or as drug loaded microvesicles and subsequent use in patients, it is suspected that DPPA may contribute to undesired immune responses elicited by the microvesicles.

The present invention, according to a second aspect, relates to a liposome solution for forming microvesicles obtained by the method as described herein, comprised of a lipid blend comprised of a first phospholipid and second phospholipid having different phase transition temperatures (Tm) dissolved in a mixture of an organic non-aqueous solvent and an organic aqueous solvent, and wherein the liposome solution has an organic non-aqueous solvent to organic aqueous solvent ratio of 5 to 25 non-aqueous: 75 to 95 vol. % aqueous, preferably 7.5 to 20 non-aqueous: 80 to 92.5 aqueous vol.-%, even more preferably 8 to 15 non-aqueous: 85 to 92 aqueous vol.-%, most preferably about 10 non-aqueous: 90 aqueous vol.-%. Remarkable in the method of present invention in view of the known methods to produce liposome solutions is that lipids are dissolved in the organic solvent at relative low temperatures, more specifically below the Tm of said lipids. In comparison to known methods, increased volumes of non-aqueous organic solvent (preferably 7.5 vol. % or higher) is pre-heated to a temperature of below the Tm of the respective phospholipid providing a relative high concentration of solvent in the lipid solution. The range of non-aqueous solvent is preferably larger than or equal to 7.5 vol. % based on the total volume of organic solvent in the liposome solution, to achieve an optimal dissolved lipid solution at the claimed temperatures.

According to a preferred embodiment, the present invention relates to the liposome solution, wherein the first phospholipid is selected from the group consisting of 1,2-dipalmitoyl-sn-glycero-3-phosphocholine (DPPC), 1,2-distearoyl-sn-glycero-3-phosphocholine (DSPC), 1,2-dierucoyl-sn-glycero-3-phosphocholine (DEPC), 1,2-dimyristoyl-sn-glycero-3-phosphocholine (DMPC), 1,2-dimyristoyl-sn-glycero-3-phosphoglycerol (DMPG), 1,2-dioleoyl-sn-glycero-3-phosphocholine (DOPC), 1,2-dioleoyl-sn-glycero-3-phosphoethanolamine (DOPE), 1,2-dioleoyl-sn-glycero-3-phospho-L-serine (DOPS), 1,2-dipalmitoyl-sn-glycero-3-phosphoglycerol (DPPG), 1,2-distearoyl-sn-glycero-3-phosphoglycerol (DSPG), egg phosphatidylcholine (EPC), and hydrogenated soybean phosphatidylcholine (HSPC), preferably DSPC, more preferably DPPC, and wherein the second phospholipid is selected from the group consisting of N-(Carbonyl-methoxypolyethylene glycol(750-10000))-1,2-distearoyl-sn-glycero-3-phosphoetanolamine (DSPE-mPEG (750-10000)), N-(Carbonyl-methoxypolyethylene glycol(1000-5000))-1,2-dipalmitoyl-sn-glycero-3-phosphoetanolamine (DPPE-mPEG(1000-5000)), preferably DPPE-mPEG2k, more preferably DSPE-mPEG5000, most preferably DPPE-mPEG5000.

According to another preferred embodiment, the present invention relates to the liposome solution, wherein phospholipids, more specifically the sum of the first and second phospholipids, are present in the final liposome solution at concentration of 1 to 25 mg/ml, preferably 5 to 20 mg/ml, more preferably 10 to 15 mg/ml.

According to a preferred embodiment, the present invention relates to the liposome solution, wherein the non-aqueous organic solvent is one or more selected from the group consisting of propylene glycol, ethylene glycol, preferably propylene glycol.

According to yet another preferred embodiment, the present invention relates to the liposome solution, wherein the aqueous organic solvent is one or more selected from the group consisting of phosphate buffer saline (PBS), buffered aqueous solution, saline solution, preferably PBS.

According to a preferred embodiment, the present invention relates to the liposome solution, wherein the liposome solution is free from glycerol, Diphenylphosphoryl azide (DPPA) and toxic carcinogenic organic solvents, such as chloroform.

The present invention, according to a further aspect, relates to microvesicles comprised of the liposome solution disclosed herein, wherein the microvesicles are monodisperse having a particle size in diameter of 1 to 8 μm, preferably 2 to 6 μm, more preferably 3 to 5 μm. Monodisperse microvesicles disclosed herein are preferably microbubbles of approximately similar size, i.e. having a uniform size distribution, having a geometric standard deviation (GSD)≤1.1.). The liposome solution of the present invention enables the formation of foam-free monodisperse lipid-coated microbubbles at low temperature having reduced coalescence tendency. Monodisperse microbubbles disclosed herein have a median diameter of below 10 μm, preferably in the range of from 2 to 6 μm, more preferably 3 to 5 μm, and a geometric standard deviation GSD preferably smaller than or equal to 1.1. Microbubbles with larger diameters (>10 μm) are undesired because they will be filtered out of the blood circulation by the lungs after an intravenous injection. Too small microbubbles (<1 μm) will be inefficient because they are acoustically overdamped and will not resonate in an ultrasound field.

According to a preferred embodiment, the present invention relates to the microvesicles, wherein said microvesicles are drug delivery enhancing agents or drug-loaded microvescicles for delivery of drugs to a patient, preferably parental delivery of drugs.

The present invention, according to a further aspect, relates to the use of a liposome solution or microvesicles disclosed herein for the drug delivery to a patient, preferably via parental administration, more preferably via intravenous co-administration.

The present invention will be further detailed in the following examples and figures wherein:

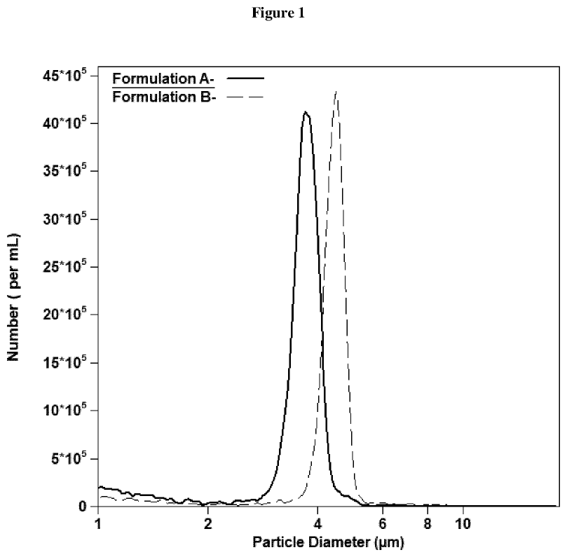

FIG. 1: shows the size distribution of monodisperse microbubbles obtained after microfluidic flow focusing formation with the liposome compositions obtained according to the method of present invention (formulation B) in comparison to microbubbles obtained via a known method in the art (formulation A).

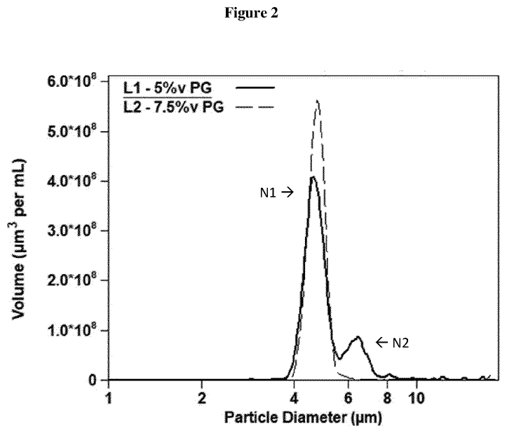

FIG. 2: shows the size distributions of monodisperse microbubbles obtained after microfluidic flow focusing formation with the liposome compositions L1 and L2 measured using a Beckman Coulter Counter Multisizer 4e. Both lipid formulations are prepared according to the method of present invention comprised of 80/20 molar percentage ratio of primary lipid (DSPC) and secondary PEGylated lipid (DPPE-mPEG5k). The total lipid concentration is 15 mg/ml. L1 has an organic non-aqueous solvent to organic aqueous solvent ratio of vol-5% Propylene glycol (PG) and vol-95% Phosphate buffered saline (PBS). L2 has an organic non-aqueous solvent to organic aqueous solvent ratio of vol-7.5% Propylene glycol (PG) and vol-92.5% Phosphate buffered saline (PBS). The L1 formulations shows multiple peaks (N1, N2) due to coalescence, whereas L2 show a single sharp peak (N1).

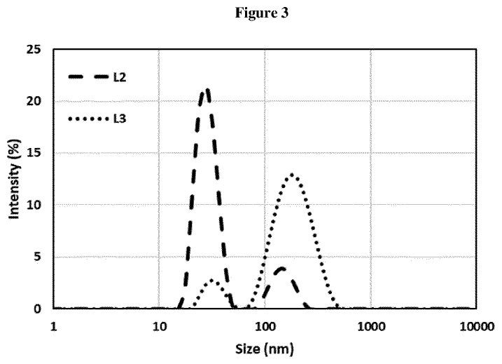

FIG. 3: shows the relative intensity based (%) size distribution of the lipid vesicles for L2 and L3 formulations (total lipid concentration of 15 mg/ml) by Dynamic Light Scattering (DLS) using the Zetasizer NanoS. The DLS measurements show that there are smaller liposomes of <100 nm dominantly present in lipid formulation L2 than in lipid formulation L3, and vice versa for the L3 formulation comprising dominantly liposomes of >100 nm.

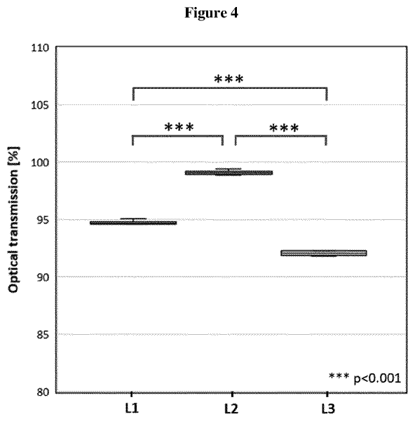

FIG. 4: shows the optical transmission (%) of the three lipid formulations L1, L2 and L3 of example 3 using the Jenway spectrophotometer 630 at 700 nm. All formulations have identical total lipid concentration of 15 mg/mL. The higher the transmission readings the smaller the lipid vesicles are in the lipid formulation. L2 shows a higher optical transmission reading than lipid formulation L1 and L3 indicating a difference in size distribution of the lipid vesicles within the three lipid solutions. L2 comprised of the smallest size lipid vesicles compared to lipid formulation L1 and L3. Because the total lipid concentration was the same for all three lipid formulations, this indicates a higher concentration of lipid vesicles in lipid formulation L2 compared to lipid formulation L1 and L3, which is beneficial for preventing, microbubble coalescence.

### EXAMPLES

### Example 1—Preparation of Liposome Formulations for Large Scale Microvesicle Formation

Methods A and B were followed for providing liposome solutions for the subsequent formation of microvesicles according to the method of present invention.

### Method A

Propylene Glycol (10 vol. %) is preheated at 38° C. Then, a first lipid DPPC having the lowest Tm is added (80 mol-%) and gently stirred for approx. 20 min until lipid dissolution. Then a second lipid DPPE-mPEG5k is added (20 mol-%) to the same vial containing the dissolved first lipid. The total lipid concentration is 15 mg/mL. The lipid solution is maintained in a water bath at 38° C. and gently stirred for approx. 1 hour. Next, preheated PBS solution at 38° C. s slowly added under stirring to obtain a 10/90% v/v Propylene Glycol/PBS lipid containing solution. Keep the liposome solution under stirring at 38° C. for about 6 hours. Next the liposome solution is flow extruded over a 0.2 μm polycarbonate/cellulose acetate membrane at 38° C. to obtain a liposome solution that enables monodisperse microvesicle formation by microfluidic flow focusing. The resulting liposome solution may subsequently be stored, preferably at 4° C.

### Method B

A first stock solution is prepared by dissolving the first lipid DPPC (80 mol-%) in preheated Propylene Glycol (10 vol. %), as described in Method A. A second stock solution is prepared separately with the second lipid DPPE-mPEG5k (20 mol-%) and add to preheated PBS at 50° C. to obtain a ~10/90 vol.-% ratio with the organic solvents when later combining the first and second stock solutions. Keep the second stock solution at 50° C., under gently stirring. Keep both stock solutions at 38° C. and 50° C., respectively and gently stir, preferably for approx. 1 hour. Then, slowly, add the second stock solution to the first stock solution, preferably stepwise in three to four steps to reduce or even prevent partial precipitation of the lipids, to obtain the liposome solution. Keep the liposome solution under stirring at 38° C. for about 6 hours to obtain a clear homogenous solution wherein the liposomes are fully dissolved. Next the liposome solution is flow extruded over a 0.2 μm polycarbonate/cellulose acetate membrane at 38° C. to obtain a liposome solution that enables monodisperse microvesicle formation by microfluidic flow focusing. The resulting liposome solution may subsequently be stored, preferably at 4° C.

### Example 2—Monodisperse Microbubble Formation by Microfluidic Flow-Focusing

Two-phase microfluidic flow focusing is a technology using a microfluidic chip in which a dispersed phase is focused by a continuous phase through a narrow orifice, where the dispersed phase experiences capillary instability and pinches off releasing monodisperse particles. In the case of using a gas as the dispersed phase (e.g., C3F8) and a liquid as the continuous phase (e.g., liposome solution), the particles consist of gas-filled monodisperse microbubbles stabilized by a lipid monolayer. Here, a microfluidic flow focusing device was used with a channel geometry as described by Segers et al. (Soft Matter 2018, 14, 9550-9561). Both the gas pressure and the liquid flow rate were controlled by pressure regulators (i.e., pressure-based flow control of liquid supply). In this way, the size and concentration of the microbubbles can be controlled by the ratio between the gas and the liquid pressures.

Monodisperse microbubbles were formed at a constant 25° C. (controlled temperature on chip) using pure C3F8 as gas (i.e., no gas mixture was used) and two liposome formulations; liposome formulations A and B. Liposome formulation A is obtained according to a state of the art method wherein Formulation A was prepared at 72° C. by using DPPC/DPPE-mPEG5k at 80/20 mol-% and a Propylene Glycol/PBS ratio of 5/95 vol.-%. Briefly, a hydrated phospholipids solvent mixture was prepared by dissolving DPPC and DPPE-mPEG5k at 72° C. in Propylene Glycol to form a dissolved phospholipid solvent mixture. Next, an aqueous phosphate buffer is added and mixed to the dissolved phospholipids solvent mixture to a hydrated phospholipids solvent mixture. Liposome formulation B is obtained according to Method A, as disclosed above, i.e., according to a method of present invention, i.e., prepared at 38° C. using DPPC/DPPE-mPEG5k at 80/20 mol-% and with a Propylene Glycol/PBS ratio of 10/90 vol.-%.

The gas pressure used for microbubble formation is 1750 mbar; the gas pressure used to control the liquid flow rate was 2015 mbar for formulation A and 2050 mbar for formulation B. The outlet of the flow focusing device was connected to a 24G needle via PEEK tubing for collecting the microbubbles in sealed glass vials; the glass vials were pre-filled with 1-mL of 0.9% NaCl and purged with C3F8 gas such that the head space was filled with C3F8. A second needle was pierced through the closing rubber stopper and was used as vent by positioning it near the bottom of the glass vials, which were placed upside-down during microbubble collection. Microbubbles were collected for 5-7 minutes. The needles were removed after microbubble formation was completed, and the vials were put to rest for 1 hour to allow the microbubbles to stabilize in the vials. After 1 hour, size distributions were measured using a Coulter Counter Multisizer 4e. FIG. 1 shows the microbubble size distribution as measured with a Coulter Counter Multisizer 4e. It shows the size distributions of the monodisperse microbubbles obtained with formulation A- (blue) and formulation B- (red). In Table 1, the size distribution statistics are listed of the monodisperse microbubble samples obtained with formulation A and B.

TABLE 1
Microbubble (MB) size distribution statistics
(GSD = geometric standard deviation).

| | MB number concentration | Median diameter | |
|---|---|---|---|
| Formulation | [×10⁶ mL⁻¹] | [μm] | GSD |
| A | 69 | 3.7 | 1.09 |
| B | 55 | 4.4 | 1.06 |

As can be conclude from FIG. 1 and the above results in Table 1, monodisperse microbubbles formed by microfluidic flow focusing can be obtained by using 100% C3F8 as a gas (no gas mixture) and a liposome solution prepared according to the method of present invention.

The method of present invention provided the desired small microbubble diameter of 3 to 5 μm with most of the produced microbubbles within this size distribution range. The microbubbles obtained with formulation B are at least comparable, even slightly improved, in comparison to the microbubbles obtained with formulation A. Microbubble coalescence and foam formation, resulting in a polydisperse size distribution, was not observed in the Coulter Counter measurement, and slightly improved for Formulation B in comparison to Formulation A.

### Example 3—Properties of Lipid Formulations

Three lipid formulations (L1 to L3) are prepared comprised of 80/20 molar percentage ratio of a primary lipid (DSPC) and a secondary PEGylated lipid (DPPE-mPEG5000);
L1 is prepared according to method A of example 1 at a temperature of ~52° C. having an organic non-aqueous solvent to organic aqueous solvent ratio of vol-5% Propylene glycol (PG) and vol-95% Phosphate buffered saline (PBS).
L2 is prepared according to method A of example 1 at a temperature of ~52° C. having an organic non-aqueous solvent to organic aqueous solvent ratio of vol-7.5% Propylene glycol (PG) and vol-92.5% Phosphate buffered saline (PBS).
L3 is prepared according to the method disclosed in WO2022139582A1 at a temperature of ~72° C. having an organic non-aqueous solvent to organic aqueous solvent ratio of vol-5% Propylene glycol (PG) and vol-95% Phosphate buffered saline (PBS).
   For all lipid formulations L1, L2 and L3 the identical total lipid concentration was used of 15 mg/ml.

As in Example 2, using a microfluidic flow focusing device monodisperse microbubbles were formed at a constant 40° C. (controlled temperature on chip) with the prepared liposome formulations. Microbubble size distributions were measured using A Beckman Coulter Counter Multisizer 4e according to manufacturer's instructions, for lipid formulations L1 and L2, see Table 2. Microbubbles formed with the L2 formulation showed a reduced microbubble coalescence in comparison to microbubbles formed with the L1 formulation (second peak in the size distribution). In fact, no coalescence is observed during microbubble formation when L2 is used. In comparison, clear coalescence is observed during microbubble formation with L1 (FIG. 2), resulting in multiple peaks.

The coalescence factor (CF) for microbubbles formed with the L1 formulation is calculated according to the equation;

$$C F = ( 2 * N 2 ) / ( N 1 + 2 * N 2 ) * 100 \qquad ( 1 )$$

With N2 the number of microbubbles in the second peak of the size distribution and N1 the number of microbubbles in the first peak in the size distribution. The number of microbubbles in the second peak (N2) is multiplied with 2 because the microbubbles that are part of the second peak were originally formed by two microbubbles that were part of the first peak. (N1+2*N2) is then equal to the total amount of microbubbles in the main peak if no coalescence would have occurred.

TABLE 2
Microbubble Coulter Counter statistics (microbubble (MB)
number concentration and median diameter) and derived coalescence
factor when using different lipid formulations.

| | MB number | Median | Coalescence |
|---|---|---|---|
| | concentration | diameter | factor (CF) |
| Formulation | [×10⁶ mL⁻¹] | [μm] | (%) |
| L1 | 130 | 4.6 | 15 |
| L2 | 145 | 4.7 | 0 |

It was further observed that the prepared lipid solution L2, using 7.5% v propylene glycol during preparation, is easier to filter in comparison to L1 when 5% v propylene glycol is used during preparation of the lipid formulation. Most likely due to a better solubility of the phospholipids in L2.

Next, size distribution of the lipid vesicles was measured in more detail for L2 and L3 by using a Zetasizer NanoS for Dynamic Light Scattering (DLS) measurements. DLS measurements provide information on the mean particle size as well as on particle size distribution. FIG. 3 is an intensity-based size distribution which was obtained by DLS measurements using the Zetasizer NanoS. This type of measurements determines the size of single particles based on the amount of light scattered. It does not give direct information about the absolute number of particles. However, since large particles scatter more light than small particles, the peak with the larger particles is often dominantly present in the intensity-based size distribution. In case the small sized particle peak is dominant it indicates that there is a high number of small particles present in the solution.

The DLS measurements show that in lipid formulation L2 there are smaller liposomes of <100 nm dominantly present in lipid formulation L3, and vice versa for the L3 formulation liposomes of >100 nm are dominantly present (FIG. 3). It is known in the art that for a fixed total lipid concentration, an increase in liposome size increases the chance on coalescence, probably due to a decrease in liposome concentration and decrease in relative viscosity. Here it is shown that a high concentration of small liposomes (<100 nm) is beneficial in view of reducing or even preventing microbubble coalescence when using microfluidic flow focusing to form microbubbles. A high concentration of small liposomes, i.e. below 100 nm, is beneficial for preventing microbubble coalescence. Furthermore, due to lower temperature needed in providing the L2 formulation in comparison to the L3 formulation, less energy is consumed during preparation of lipid formulation L2 compared to L3.

Finally, the optical transmission was measured using a Jenway spectrophotometer 630 that measures the amount of light (700 nm) transmitted through the lipid solution compared to a reference measurement. The reference measurement was done on phosphate buffered saline (PBS) which is the main component of the lipid solution. The optical transmission reading of the reference sample is set at 100%. The closer the optical transmission reading is to 100% the more equally transparent the sample is to the reference measurement. An optical transmission reading of below 100% indicates that the loaded sample has absorbed/scattered some of the incoming light. Also in this case, smaller particles scatter less resulting in a higher optical transmission reading, and vice versa, larger particles scatter more resulting in lower optical transmission reading Although the three lipid formulations L1, L2 and L3 have the same total lipid concentration of 15 mg/mL, lipid formulation L2 shows a higher optical transmission reading than lipid formulation L1 and L3 (FIG. 4). This implies a difference in size of the lipid vesicles within the three lipid solutions. In addition, it implies that due to the higher transmission readings that there are smaller lipid vesicles present in lipid formulation L2 compared to lipid formulation L1 and L3, which is as stated before, beneficially for preventing microbubble coalescence.

1-23. (canceled)

24. A method for providing a liposome solution for formation of microvesicles, wherein the liposome solution is comprised of a phospholipid blend of at least two phospholipids having different phase transition temperatures (Tm), comprising the steps of:
a) dissolving a first phospholipid having the lowest Tm in a pre-heated organic non-aqueous solvent having a temperature of below the Tm of the first phospholipid, providing a first liposome solution,
b) adding a second phospholipid in said first liposome solution to obtain a liposome dispersion, or
adding a second phospholipid in a pre-heated organic aqueous solvent having a temperature of below the Tm of the second phospholipid to obtain a second liposome dispersion,
c) maintaining the liposome dispersion at the temperature of below the Tm of the first phospholipid for 30 min to 2 hours to obtain a further liposome solution wherein the phospholipids are dissolved; or
maintaining the second liposome dispersion at the temperature of below the Tm of the second phospholipid for 30 min to 2 hours to obtain a second liposome solution wherein the phospholipids are dissolved;
d) adding of a preheated organic aqueous solvent having a temperature of below the Tm of the first phospholipid to the further liposome solution, or
adding the second liposome solution to the first liposome solution;
to obtain a pre-final liposome solution having an organic non-aqueous solvent to organic aqueous solvent ratio of 7.5 to 20 non-aqueous: 80 to 92.5 aqueous vol.-%;
e) maintaining the pre-final liposome solution at the temperature of below the Tm of the first phospholipid for 2 to 12 hours, to obtain a final liposome solution.

25. The method according to claim 24, wherein the method further comprises step f) flow extrusion or filtering of the final liposome solution at a temperature below the Tm of the first phospholipid or at 15 to 40° C. thereby providing a final liposome solution for formation of microvesicles.

26. The method according to claim 24, wherein the temperature of the pre-heated organic non-aqueous or aqueous solvents is at most 20% or at most 10% below the respective Tm values of said first or second phospholipids.

27. The method according to claim 24, wherein the pre-heated organic non-aqueous solvent having a temperature of below the Tm of the first phospholipid in step a and the preheated organic aqueous solvent having a temperature of below the Tm of the first phospholipid in step d is 20 to 55° C.

28. The method according to claim 24, wherein the pre-heated organic aqueous solvent having a temperature of below the Tm of the second phospholipid in step b is 40 to 60° C.

29. The method according to claim 24, wherein addition of said preheated organic aqueous solvent or said second liposome solution in step d is performed in two to five separate steps, wherein the added volume is divided equally among these steps.

30. The method according to claim 24, wherein the final liposome solution comprises from 60 to 95 mol-% of the first phospholipid, or from 70 to 90 mol-%.

31. The method according to claim 24, wherein the final liposome solution comprises from 5 to 40 mol-% of the second phospholipid, or from 10 to 30 mol-%.

32. The method according to claim 24, wherein the first phospholipid is selected from the group consisting of 1,2-dipalmitoyl-sn-glycero-3-phosphocholine (DPPC), 1,2-distearoyl-sn-glycero-3-phosphocholine (DSPC), 1,2-dierucoyl-sn-glycero-3-phosphocholine (DEPC), 1,2-dimyristoyl-sn-glycero-3-phosphocholine (DMPC), 1,2-dimyristoyl-sn-glycero-3-phosphoglycerol (DMPG), 1,2-dioleoyl-sn-glycero-3-phosphocholine (DOPC), 1,2-dioleoyl-sn-glycero-3-phosphoethanolamine (DOPE), 1,2-dioleoyl-sn-glycero-3-phospho-L-serine (DOPS), 1,2-dipalmitoyl-sn-glycero-3-phosphoglycerol (DPPG), 1,2-distearoyl-sn-glycero-3-phosphoglycerol (DSPG), egg phosphatidylcholine (EPC), and hydrogenated soybean phosphatidylcholine (HSPC).

33. The method according to claim 24, wherein the second phospholipid is a phospholipid poly ethylene glycol (PEG) conjugate, selected from the group consisting of N-(Carbonyl-methoxypolyethylene glycol (750-10000))-1,2-distearoyl-sn-glycero-3-phosphoetanolamine (DSPE-mPEG(750-10000)), N-(Carbonyl-methoxypolyethylene glycol(1000-5000))-1,2-dipalmitoyl-sn-glycero-3-phosphoetanolamine (DPPE-mPEG(1000-5000)).

34. The method according to claim 24, wherein phospholipids, more specifically the sum of the first and second phospholipids, are present in the final liposome solution at concentration of 1 to 25 mg/ml, or 5 to 20 mg/ml.

35. The method according to claim 24, wherein the non-aqueous organic solvent is one or more selected from the group consisting of propylene glycol, ethylene glycol.

36. The method according to claim 24, wherein the aqueous organic solvent is one or more selected from the group consisting of phosphate buffer saline (PBS), buffered aqueous solution, saline solution.

37. The method according to claim 24, wherein the method or liposome solution is free from glycerol or Diphenylphosphoryl azide (DPPA) and toxic carcinogenic organic solvents, such as chloroform.

38. A liposome solution obtained by the method of claim 24 for forming microvesicles, comprised of a lipid blend comprised of a first phospholipid and second phospholipid having different phase transition temperatures (Tm) dissolved in a mixture of an organic non aqueous solvent and an organic aqueous solvent, and wherein the liposome solution has an organic non-aqueous solvent to organic aqueous solvent ratio of 7.5 to 20 non-aqueous: 80 to 92.5 aqueous vol.-%.

39. The liposome solution according to claim 38, wherein the first phospholipid is selected from the group consisting of 1,2-dipalmitoyl-sn-glycero-3-phosphocholine (DPPC), 1,2-distearoyl-sn-glycero-3-phosphocholine (DSPC), 1,2-dierucoyl-sn-glycero-3-phosphocholine (DEPC), 1,2-dimyristoyl-sn-glycero-3-phosphocholine (DMPC), 1,2-dimyristoyl-sn-glycero-3-phosphoglycerol (DMPG), 1,2-dioleoyl-sn-glycero-3-phosphocholine (DOPC), 1,2-dioleoyl-sn-glycero-3-phosphoethanolamine (DOPE), 1,2-dioleoyl-sn-glycero-3-phospho-L-serine (DOPS), 1,2-dipalmitoyl-sn-glycero-3-phosphoglycerol (DPPG), 1,2-distearoyl-sn-glycero-3-phosphoglycerol (DSPG), egg phosphatidylcholine (EPC), and hydrogenated soybean phosphatidylcholine (HSPC), and/or wherein the second phospholipid is selected from the group consisting of N-(Carbonyl-methoxypolyethylene glycol(750-10000))-1,2-distearoyl-sn-glycero-3-phosphoetanolamine (DSPE-mPEG(750-10000)), N-(Carbonyl-methoxypolyethylene glycol (1000-5000))-1,2-dipalmitoyl-sn-glycero-3-phosphoetanolamine (DPPE-mPEG(1000-5000)).

40. The liposome solution according to claim 38, wherein the sum of the first and second phospholipids, are present in the final liposome solution at concentration of 1 to 25 mg/ml or 5 to 20 mg/ml.

41. The liposome solution according to claim 38, wherein the non-aqueous organic solvent is one or more selected from the group consisting of propylene glycol, ethylene glycol, and/or wherein the aqueous organic solvent is one or more selected from the group consisting of phosphate buffer saline (PBS), buffered aqueous solution, saline solution, PBS.

42. The microvesicles comprised of the liposome solution of claim 38, wherein the microvesicles are monodisperse having a particle size in diameter of 1 to 8 μm, or 2 to 6 μm, wherein monodisperse microvesicles have a uniform size distribution comprising a geometric standard deviation (GSD)≤1.1 and/or wherein said microvesicles are drug delivery agents or drug-loaded microvesicles for delivery of drugs to a patient, parental delivery of drugs.

43. A use of a liposome solution of claim 38 or microvesicles comprised of said liposome solution for the drug delivery to a patient, via parental administration or intravenous administration.
